(19) United States
(12) Patent Application Publication
(10) Pub. No.: US 2017/0259089 A1
(43) Pub. Date: Sep. 14, 2017

(54) HELMET INCLUDING AIR CIRCULATION SYSTEM

(71) Applicant: Carlos M. De Jesus, Sun Valley, CA (US)

(72) Inventor: Carlos M. De Jesus, Sun Valley, CA (US)

(21) Appl. No.: 15/451,658

(22) Filed: Mar. 7, 2017

Related U.S. Application Data
(60) Provisional application No. 62/389,836, filed on Mar. 11, 2016.

(51) Int. Cl. A62B 18/04; A61M 16/06; A61F 9/06

(52) U.S. Cl. CPC A62B 18/045; A61F 9/068; A61M 16/06; A61M 2205/3306; A61M 2205/3368; A61M 2205/8293; A61M 2205/50

(57) ABSTRACT

A helmet includes a mask and an air circulation system mounted on the mask, the air circulation system comprising an air blower and a processing system, electronically coupled to the air blower, the processing system comprising a controller, wherein the controller is to receive a first signal representing a light intensity detected by a photo sensor, determine initialization of a welding gun in view of the first signal, and responsive to determine the initialization of the welding gun, issue a first instruction to start an operation of the air blower.

### RELATED APPLICATION

The present application claims benefit from U.S. Provisional Patent Application No. 62/389,836, entitled “Air Flow Fan System,” filed on Mar. 11, 2016, the content of which is hereby incorporated by reference in its entirety.

### TECHNICAL FIELD

This disclosure relates to a helmet and, in particular to, a helmet including an air circulation system.

### BACKGROUND

A welding gun in operation can generate intensive heat and toxic fumes which may include gas, smoke and vapor harmful to the operator's health. The operator (i.e., the person who operates the welding gun) may wear a welding helmet to protect the operator from direct exposure to the heat and sparks generated during the welding process. The generated fumes, however, may find their way to enter the interior of the welding helmet and be trapped in the space between operator's face and the inner surface of the welding helmet.

### BRIEF DESCRIPTION OF THE DRAWINGS

The disclosure is illustrated by way of example, and not by way of limitation, in the figures of the accompanying drawings.

FIG. 1 illustrates a helmet including an air circulation system according to an implementation of the disclosure.

FIG. 2 shows the components of a processing device according to an implementation of the disclosure.

FIGS. 3A-3D illustrate different views of the helmet according to implementations of the disclosure.

FIGS. 4A-4E illustrate an attachable air circulation system according to an implementation of the disclosure.

FIG. 5 depicts a flow diagram of a method to control an air circulation system in a helmet according an implementation of the disclosure.

### DETAILED DESCRIPTION

An air circulation system is needed to expel the toxic fumes from the space inside the welding helmet during a welding operation. Implementations of the disclosure may include a helmet (e.g., a welding helmet) that includes an air circulation system to provide a constant air flow inside and outside of the helmet and to expel toxic fumes generated in a proximity of the helmet (e.g., by a welding gun operated near the helmet). The constant air flow may provide a cooling effect to the helmet wearer's face, help maintain cleaner lenses mounted on the helmet, and provide a cleaner air for the helmet wearer to breath.

In one implementation, the air circulation system may include an air blower and a processing device to control the operations of the air blower. The operations of the air blower may include a start operation, a stop operation, and a change speed operation. The air blower may be powered by rechargeable batteries. The processing device may include a programmable processor (e.g., a micro controller) and an interface to receive data from different sensors, where these sensors can be part of the helmet or accessory devices to the helmet. The processing device may receive data from these sensors via the interface and generate control instructions to the air blower. The control instructions may control the operation of the air blower, where execution of the control instructions may cause the air blower to start or to stop, or change the air exchange rate of the air blower. The air circulation system may further include at least one intake port and at least one exhaust port, flexible tubes coupled to the intake port and the exhaust port.

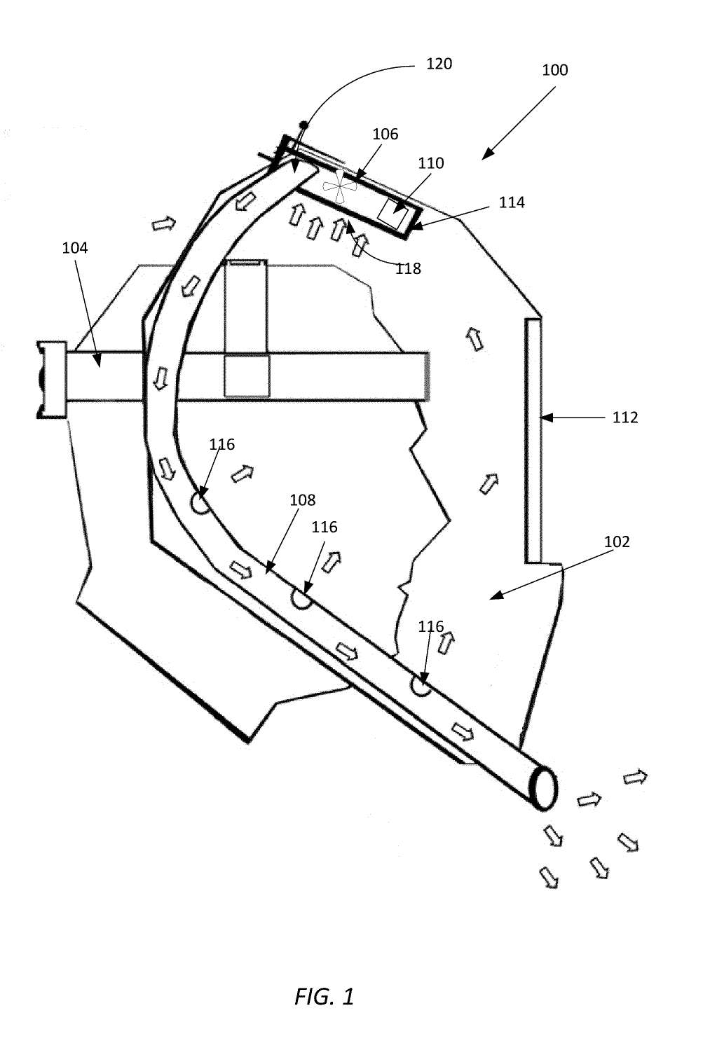

FIG. 1 illustrates a helmet 100 including an air circulation system according to an implementation of the disclosure. As shown in FIG. 1, helmet 100 may include a mask 102, a mounting component 104, flexible tube 108, and an air circulation system 114. The air circulation system 114 may further include an air blower 106 and a processing device 110. Mask 102 can be any type of mask used on a welding helmet to cover and protect the face of the wearer of the helmet from the heat and sparks during welding operations. The wearer of the helmet may be referred to as the “operator” herein. However, a wearer of the helmet does not have to be operating a tool, such as a welding gun, when using the helmet described herein. Mask 102 may include a lens 112 made from transparent materials through which the operator may observe the welding gun operation. Mounting component 104 attached to mask 102 may include straps to mount the helmet 100 onto the operator's head.

In one implementation, air circulation system 114 may include a casing (not shown), an air blower (e.g., a fan) 106, and a power source (not shown) (e.g., a rechargeable battery or a lithium battery). The air blower 106 may be electrically connected to the power source via a switch device. The casing may be made of solid materials (e.g., metals or plastics). The casing may enclose the air blower and include an input outlet 118 and an exhaust outlet 120. A first end of flexible tube 108 is coupled to the casing of air blower 106 via the exhaust outlet 120, and a second end of flexible tube 108 is extended to outside of the helmet. For example, the second end of flexible tube 108 may be extended from the back of the user's head to the front.

In one implementation, flexible tube 108 may be perforated with holes 116. The holes 116 may be located at different positions on tube 108. The positions of holes 116 are designed to facilitate the air flow. For example, holes 116 may be placed along the side of helmet 100 near the nose and mouth of the operator. When the air blower 106 is turned on, the air trapped in the helmet may be pulled by the air blower 106 into the casing via the input outlet. Air circulation system 114 may include an air filter (not shown) for purifying the air drawn into the air circulation system 114. The purified air may then be pushed out into the flexible tube through the exhaust port. A portion of the purified air may be pushed outside helmet 100, and a small portion of the purified air may be pushed through holes 116 back into inside of helmet 100 for operator to breath. Thus, the air (including harmful fumes) trapped in the helmet 100 may be circulated in exchange for purified air.

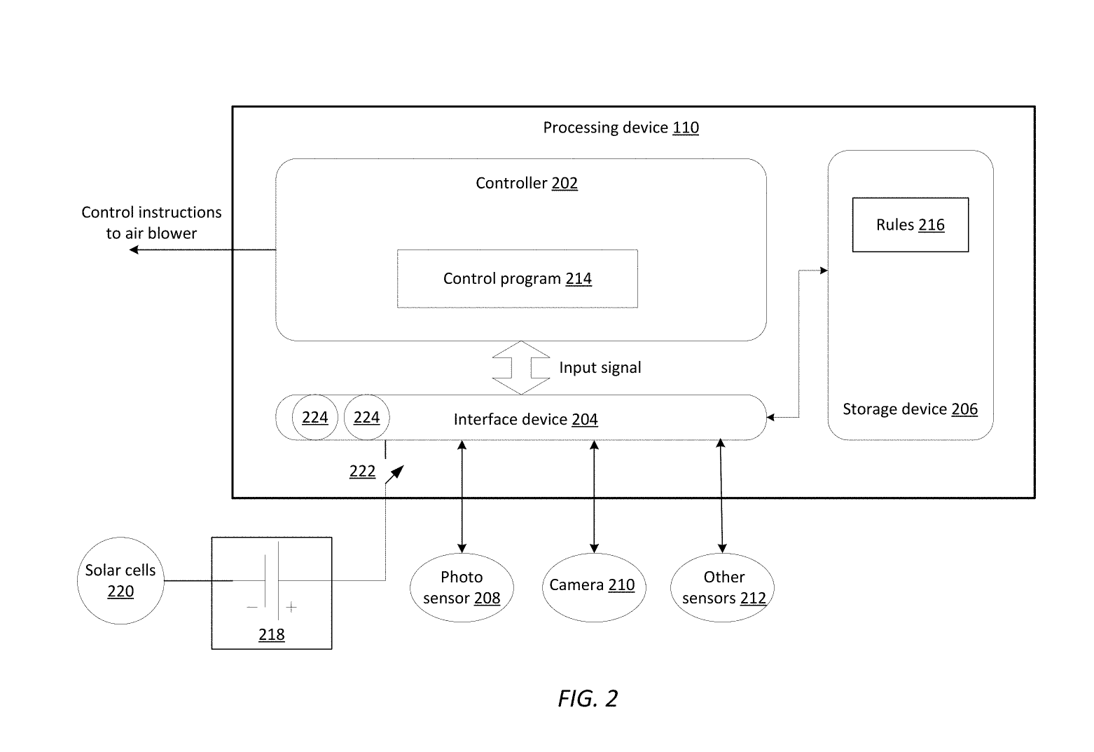

Processing device 110 may include a number of hardware components electronically connected for controlling the operation of the air blower 106. FIG. 2 shows the components of processing device 110 according to an implementation of the disclosure. As shown in FIG. 2, processing device 110 may include a controller 202, an interface device 204, and an optional storage device 206. Controller 202 can be a programmable logic controller (PLC) that may be programmed with a control program 214 in the form of machine-executable code. Interface device 204 may receive input signals from multiple sensors 208, 210, 212, and convert the input signals into forms that can be read by controller 202.

Interface device 204 may include ports to receive different types of input signal. For example, a first port may be designated to receive the input signal from sensor 208; a second port may be designated to receive the input signal from sensor 210; a third port may be designated to receive the input signal from sensor 212. These ports can be a 5-volt type of ports such as, for example, a Universal Serial Port (USB) port, or a MICRO port. In one implementation, interface device 204 may include an analog-to-digital converter (ADC) that may convert analog signals received from sensors 208, 210, 212 into digital signals (e.g., binary data) that can be processed by a digital controller (e.g., a digital signal processor (DSP)). Control program 214 may process the input signals and generate control instructions for air blower 106. The control instructions may control the operations of air blower 106, including a start instruction to start the air blower, a stop instruction to stop the air blower, and a speed instruction to control the speed of a fan in the air blower.

In one implementation, interface device 204 may provide one or more 5-volt connectors 224 for connecting processing device 110 with electronic accessory devices. For example, processing device 110 may provide 5-volt electrical supply to at least one of a flash light or a speaker. The flash light may be used to illuminate the working area of the operator, and the speaker may be used by the operator to communicate with other persons (e.g., co-workers).

In one implementation, processing device 110 may optionally include a storage device 206 to store programming code and data. Storage device 206 can be register devices and/or memory device (e.g., random access memory (RAM)). For example, storage device 206 may store the executable code and associated parameters of control program 214 and may also store data collected by sensors 208, 210, 212.

In one implementation, interface device 204 may be communicatively connected to a photo sensor 208. Photo sensor 208 is a photo-electronic device that detects the intensity of light shining on a sensing element of the photo sensor 208. The light can be visible light, infrared light, and ultraviolet light. Responsive to detecting the light shined on photo sensor 208, photo sensor 208 may generate an electronic signal with a power (as a function of amplitude of the light wave) corresponding to the intensity of the light. Interface device 204 may capture the electronic signal representing the light shined on phone sensor 208 and convert the electronic signal into digital signals for controller 202 to further process.

Responsive to receiving the electronic signal representing the light intensity changes over time detected by photo sensor 208, controller 202 may execute control program 214 to analyze the light intensity changes and detect certain trigger events.

Control program 214 may identify a pattern in the light intensity change representing the ignition of a welding gun. In one implementation, control program 214 may detect a sharp light intensity change representing from a dim light to a very bright light (e.g., the jump in the light intensity is greater than a threshold value), where the event may represent the lightening up of the welding gun triggered by the operator. Responsive to detecting the event representing the lighting up of the welding gun, control program 214 may generate a start instruction to air blower 106 to start the air circulation system 100. In one implementation, control program 214 may issue the start instruction to air blower 106 immediately responsive to detecting that the welding gun has started. In another implementation, control program 214 may delay (e.g., five seconds after the detection of welding gun ignition) the issuance of the start instruction to the air blower 106.

In another implementation, control program 214 may detect a sharp light intensity change representing from a very bright light to a dim light, where the event may represent a shutdown of the welding gun. Responsive to detecting the event representing the shutdown of the welding gun, control program 214 may generate a stop instruction to air blower 106 to stop the air circulation system 100. In one implementation, control program 214 may issue the stop instruction to air blower 106 immediately after detecting that the welding gun has stopped. In another implementation, control program 214 may delay (e.g., five seconds after the detection of welding gun ignition) the issuance of the stop instruction to the air blower 106 to give air blower 106 extra time to exhaust residual fumes in the welding helmet. In one implementation, the amount of delay before starting (or stopping) the air blower 106 after the detection of an event can be set by the operator through interface device 204. For example, interface device 204 may include a number of choices (e.g., one second, two seconds, and five seconds) for the operator to choose. Interface device 204 may include elements (e.g., buttons) corresponding to these choices for the operator to select.

In one implementation, interface device 204 may be communicatively connected to a camera 210 that is mounted on the head of the user. Camera 210 may include a photo sensor (as photo sensor 208) that may detect events representing the start and/or stop of the welding gun. Additionally, camera 210 may be mounted with its lens towards the welding gun to record the process of welding. Alternatively, interface device 204 may include a wired and/or wireless communication link (e.g., a Bluetooth® link) to transmit the recoding to a remote storage device. The recording may be stored in storage device 206 associated with processing device 110. In one implementation, the detection of the welding gun start may trigger the start of the video recording, and the detection of the welding gun stop may cause the stop of the video recording. The recorded video may be used for later quality control and training purpose.

In one implementation, interface device 204 may be communicatively connected to other types of sensors (e.g., one or more sensors 212) that may measure different aspects of the welding environment. For example, sensors 212 may include different types of environmental sensors that measure an environmental quantity, where the environmental quantity is a value representing a physical aspect of the working environment. In one implementation, sensors 212 may include a temperature sensor that records the temperatures during the welding and an air quality sensor to measure an air quality quantity (e.g., a particle density). Sensors 212 may also include chemical sensors that may monitor chemical elements (e.g., chromium, nickel, arsenic, manganese etc.) in the air through the welding process. The temperature sensor and the chemical sensors may transmit the physical and chemical quantities they measure to interface device 204 which may convert these quantities to electronic signals for controller 202. Controller 202 may execute control program 214 to record these environmental quantities in storage device 206 and analyze these quantities to determine whether the welding environment meets pre-defined safety rules 216 stored in storage device 206. For example, in one implementation, storage device 206 may store a set of rules 216 that are prescribed according to safety regulations mandated by the government or the company. The safety regulations may specify the duration and/or the amount harmful chemicals to which an operator can expose. Control program 214 may continuously receive measured quantities from the interface device 204 and compare the measured quantities with the rules 216 to determine whether the welding environment violates one or more safety regulations.

In one implementation, responsive to detecting a violation of the safety regulations based on the measured quantities and rules 216, controller 202 may cause to generate a warning signal to the user. The warning signal may inform the operator about the detected violation. The warning signal can be an audio warning (e.g., a buzz sound) or a visual warning (e.g., a red indicator connected to the processing device 110). In one implementation, the detection of a certain violation may cause the controller 202 to issue a speed instruction to the air blower 106 that may increase the air blower speed and increase the volume of air circulation.

Sensors 212 may also include an acoustic sensor (e.g., a microphone) that may receive the audio during the welding. The operator may talk to the microphone and record audio clips as annotations of the welding operation.

In one implementation, processing device 110 may be powered by a rechargeable battery pack 218. Battery pack 218 may provide, via interface device 204, the power supply to processing device 110. In one implementation, one or more solar panels 220 may be electronically connected to rechargeable battery pack 218. The one or more solar panels 220 may charge the rechargeable battery pack 218 during daylight. In one implementation, processing device 110 may include an ON/OFF switch 222 coupled between batteries 218 and interface device 204. Operator may use switch 222 to manually turn on or off the air circulation system 114. In one implementation, switch 222 may be implemented with a large button (e.g., approximately, an inch by an inch) to allow the operator manually turn on or off air circulation system even when the operator wears heavy gloves.

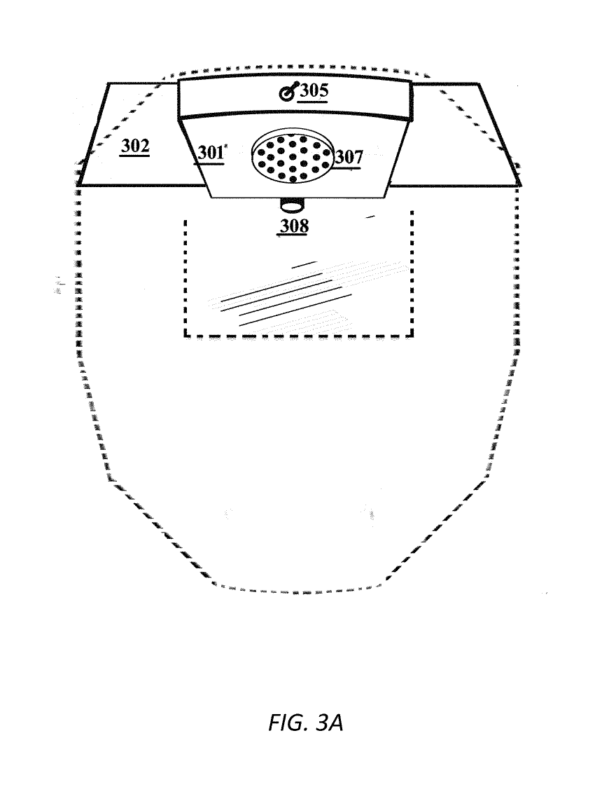

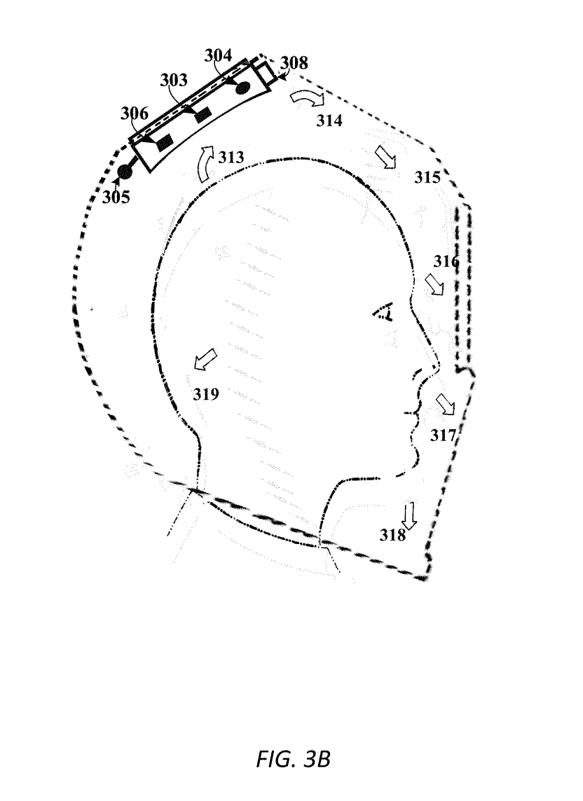

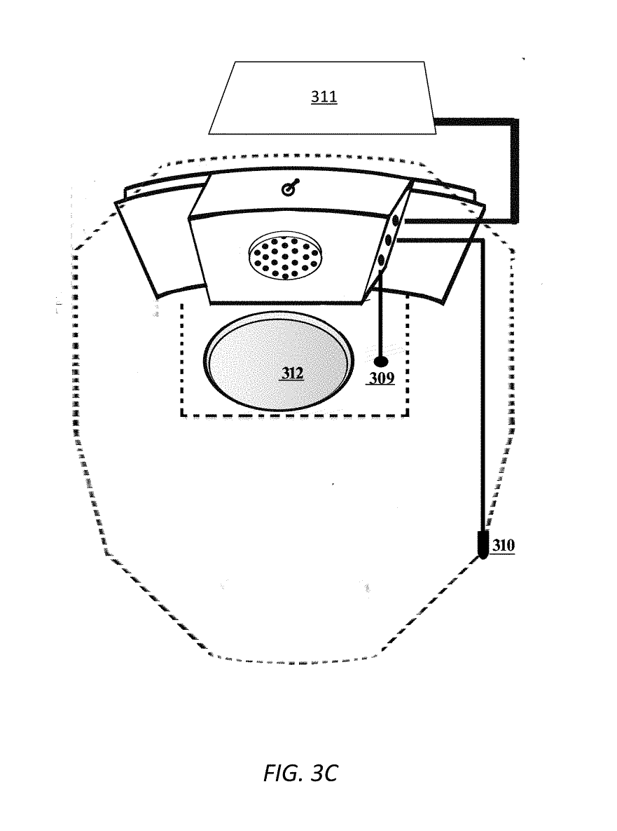

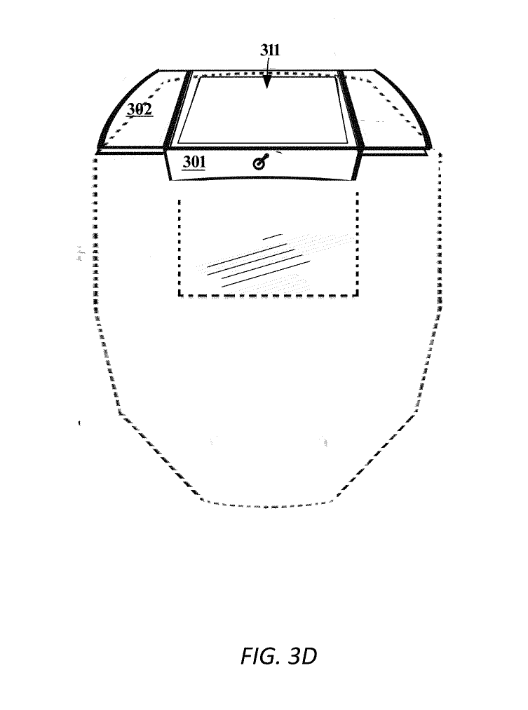

In one implementation, the processing device 110 and air blower 106 are compact devices that can be mounted on the helmet 100. FIGS. 3A-3D illustrate views of components of processing device 110 on helmet 100 according to implementations of the disclosure. FIG. 3A is a front view of the helmet 100 (as shown in FIG. 1) according to an implementation of the disclosure. Referring to FIG. 3A, air circulation system 301 may be mounted on the inner surface toward the top of the helmet. In one implementation, air circulation system 301 may be enclosed in a curved case. The case may be made from a solid material such as, for example, plastic or metal. In one implementation, the outer surface of the case may have a radius that matches the inner cursive surface of helmet 100. For example, the outer surface of the case may have a radius of approximately six inches that matches most inner surface of helmets.

Air circulation system 301 may be mounted on a flap 302 that is clamped to the rear of the helmet. As shown in FIG. 3A, air circulation system 301 may include an ON/Off switch 305, an inlet air duct 307, and an output air duct 308. In one implementation, the inlet air duct 307 may include an attachment bracket to hold an air filter. The air filter may be used to remove a substantial portion of harmful elements in the air passing through the filter. Output air duct 308 may include an optional hose attached to a flexible tube to direct the outgoing air to specific areas within the helmet.

In one implementation, the air duct 307 may include a hook to receive an air filter. Air duct 307 may include a female track to a hook of the air filter through which the air filter may be mounted onto the air duct 307.

FIG. 3B is a side view of the helmet 100 (as shown in FIG. 1) according to an implementation of the disclosure; FIG. 3C is a back view of the helmet 100 (as shown in FIG. 1) according to an implementation of the disclosure. Referring to FIGS. 3B and 3C, air circulation system 301 may further include ports 303, 304, 306 (e.g., 5-volt connectors). Port 303 is to receive a connection link to a photo sensor 310 which provides information about the activation of the weld arc. As discussed above, the air circulation system 301 can be turned on and off automatically (e.g., without user or operator intervention) based on the provided information. This feature may save the battery by switching off air circulation system 301 automatically. Port 304 is to be connected to a variety of electronic devices such as, for example, a camera 309, a flash light, and/or a speaker. Port 306 is to connect to a power supply, which may be a rechargeable battery charged by solar panels. Further, processing device 301 may be coupled to an air blower (not shown) and issue instructions that control the operation of the air blower. Air blower 308 may be enclosed in a casing that has output air duct 307 that may be coupled to flexible tubes.

FIG. 3B also illustrates the air flow patterns 313-319 within helmet 100 when air circulation system 310 is turned on. At 313, air is shown to flow into the inlet air duct. At 314, air is shown to flow out of the output air duct. Labels 315-319 show the air flow direction and accumulation of air. The accumulation of air may build up pressure within helmet 100, where the pressure may help expel harmful fumes to the outside of the helmet.

Referring to FIG. 3C, a power supply (e.g., a rechargeable battery) may be mounted on the helmet and supply electrical power to air circulation system 301. A solar panel 311 may be mounted on top of power supply. Solar panel 311 may convert light into electrical power to charge power supply. In one implementation, solar panel 311 may be mounted on a top surface of the casing of air circulation system 301 as shown in FIG. 3D. The power supply may allow the air circulation system 301 to power up other electronic devices attached to the system for the WiFi capability and for media, educational, and recreational purposes. For example, camera 309 can be a WiFi camera or a wired camera to transmit, in real time, the visual of what the operator is welding. Photo sensor 310 (or smoke sensor, or motion sensor) can be coupled to air circulation system 301 to serve as a sensor for the automatic ON/OFF switch to air circulation system 301.

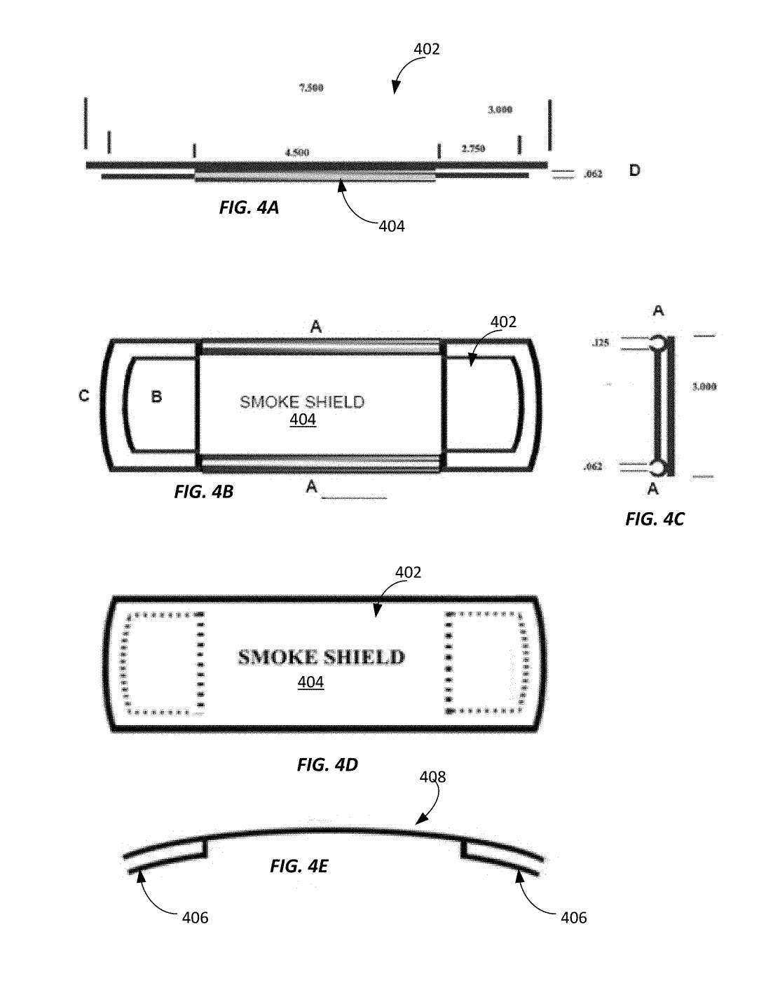

In one implementation, the air circulation system may be a separate unit that can be mounted onto different positions (e.g., the inner top section, the inner bottom section). FIGS. 4A-4E illustrates an air circulation system 402 mounted at the top rear of the helmet with a smoke shield 404 attached according to an implementation of the disclosure. FIG. 4A is a top view illustrating the relative positions of air circulation system 402 and smoke shield 404; FIG. 4B is a front view illustrating the relative positions of air circulation system 402 and smoke shield 404; FIG. 4D is a rear view illustrating the relative positions of air circulation system 402 and smoke shield 404. FIG. 4C is a cross-section view (A-A) as seen from FIG. 4B. FIG. 4E shows a clip 408 on air circulation system 402 that can be used to attach the system onto a helmet. Clip 408 may further include tracks 406 for attaching smoke shield 404.

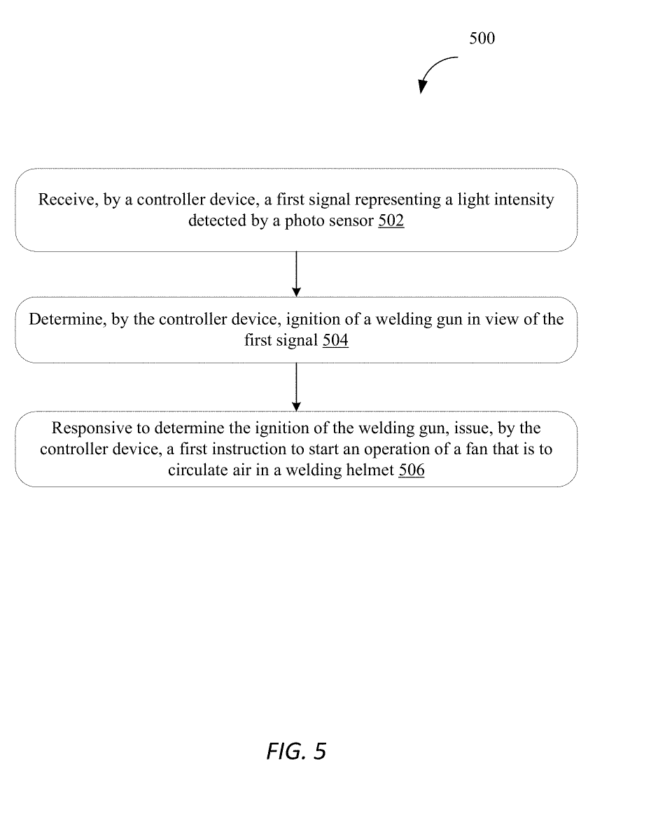

FIG. 5 depicts a flow diagram of a method 500 to control an air circulation system in a helmet according an implementation of the disclosure. Method 500 may be performed by processing devices that may comprise hardware (e.g., circuitry, dedicated logic), computer readable instructions (e.g., run on a general purpose computer system or a dedicated machine), or a combination of both. Method 500 and each of its individual functions, routines, subroutines, or operations may be performed by one or more processors or controller device of the computer device executing the method.

For simplicity of explanation, the methods of this disclosure are depicted and described as a series of acts. However, acts in accordance with this disclosure can occur in various orders and/or concurrently, and with other acts not presented and described herein. Furthermore, not all illustrated acts may be needed to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term “article of manufacture,” as used herein, is intended to encompass a computer program accessible from any computer-readable device or storage media. In one implementation, method 500 may be performed by controller 202 as shown in FIG. 1.

As shown in FIG. 5, at 502, the controller may receive a first signal representing a light intensity detected by a photo sensor.

At 504, the controller may determine ignition of a welding gun in view of the first signal.

At 506, the controller may issue a first instruction to start an operation of an air blower that is to circulate air in a welding helmet in response to determining the ignition of the welding gun.

In the foregoing description, numerous details are set forth. It will be apparent, however, to one of ordinary skill in the art having the benefit of this disclosure, that the disclosure may be practiced without these specific details. In some instances, well-known structures and devices are shown in block diagram form, rather than in detail, in order to avoid obscuring the disclosure.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” or “an implementation” or “one implementation” throughout is not intended to mean the same implementation or implementation unless described as such.

Reference throughout this specification to “one implementation” or “an implementation” means that a particular feature, structure, or characteristic described in connection with the implementation is included in at least one implementation. Thus, the appearances of the phrase “in one implementation” or “in an implementation” in various places throughout this specification are not necessarily all referring to the same implementation. In addition, the term “or” is intended to mean an inclusive “or” rather than an exclusive “or.”

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. A helmet comprising:
a mask; and
an air circulation system mounted on the mask, the air circulation system comprising:
an air blower; and
a processing system, electronically coupled to the air blower, the processing system comprising a controller, wherein the controller is to:
receive a first signal representing a light intensity detected by a photo sensor;
determine initialization of a welding gun in view of the first signal; and
responsive to determining the initialization of the welding gun, issue a first instruction to start an operation of the air blower.

2. The helmet of claim 1, wherein the air circulation system comprises a casing that encloses the air blower and the processing system, wherein the casing comprises an outlet coupled to a tube, and wherein the air blower, when started, is to generate an air pressure to expel an amount of air from inside of the mask to outside of the mask.

3. The helmet of claim 1, wherein the controller is further to:
determine termination of operation of the welding gun in view of the first signal; and
responsive to determining the termination of operation of the welding gun, issue a second instruction to stop the operation of the air blower.

4. The helmet of claim 3, wherein the first instruction is to cause the air blower to start after a first delay of time.

5. The helmet of claim 3, wherein the second instruction is to cause the air blower to stop after a second delay of time.

6. The helmet of claim 1, wherein the controller is further to:
issue a third instruction to a camera to start a recording by the camera, wherein the camera is to record an operation of the welding gun.

7. The helmet of claim 6, wherein the controller is to:
receive, from the camera, a second signal comprising a recording of the operation of the welding gun; and
store the recording in a storage device associated with the controller.

8. The helmet of claim 1, wherein the controller is to:
receive a third signal representing a measurement of an environmental quantity; and
record the third signal in the storage device.

9. The helmet of claim 8, wherein the environmental quantity comprises at least one of a temperature or an air quality quantity.

10. The helmet of claim 8, wherein the controller is to issue a fourth instruction to the air blower to change an operation status of the air blower in view of the measurement of the environmental quantity.

11. The helmet of claim 10, wherein to change the operation status of the air blower comprises one of: to increase a speed of the air blower or to decrease the speed of the air blower.

12. The helmet of claim 1, further comprises:
a power supply to provide an electrical power to the air circulation system; and
a solar panel, electronically coupled to the power supply, to charge the power supply.

13. An air circulation system, attachable to a helmet, comprising:
an air blower; and
a processing system, electronically coupled to the air blower, the processing system comprising a controller, wherein the controller is to:
receive a first signal representing a light intensity detected by a photo sensor;
determine initiation of a tool in view of the first signal; and
responsive to determining the initiation of the tool, issue a first instruction to start an operation of the air blower.

14. The air circulation system of claim 13, wherein the controller is further to:
determine termination of operation of the tool in view of the first signal; and
responsive to determining the termination of the operation of the tool, issue a second instruction to stop the operation of the air blower.

15. The air circulation system of claim 13, wherein the controller is further to:
issue a third instruction to a camera to start a recording by the camera, wherein the camera is to record the operation of the tool.

16. The air circulation system of claim 13, further comprising:
a power supply to provide an electrical power to the air circulation system; and
a solar panel, electronically coupled to the power supply, to charge the power supply.

17. A method comprising:
receiving, by a controller device, a first signal representing a light intensity detected by a photo sensor;
determining, by the controller device, initiation of a welding gun in view of the first signal; and
responsive to determining the initiation of the welding gun, issuing, by the controller device, a first instruction to start an operation of an air blower that is to circulate air in a welding helmet.

18. The method of claim 17, further comprising:
determining termination of operation of the tool in view of the first signal; and
responsive to determining the termination of the operation of the tool, issuing a second instruction to stop the operation of the air blower.

19. The method of claim 17, further comprising:
issuing a third instruction to a camera to start a recording by the camera, wherein the camera is to record the operation of the tool.

20. The method of claim 17, further comprising:
receiving a third signal representing a measurement of an environmental quantity; and
recording the third signal in a storage device.
